(19) United States
(12) Patent Application Publication
(10) Pub. No.: US 2021/0128265 A1
(43) Pub. Date: May 6, 2021

(54) Real-Time Ultrasound Imaging Overlay Using Augmented Reality

(71) Applicant: ViT, Inc., Miami, FL (US)

(72) Inventors: William Hui Jin, Miami, FL (US); Ben Bunsreng Heng, Brighton, MA (US); Richard S. Tannenbaum, New York, NY (US)

(21) Appl. No.: 17/091,084

(22) Filed: Nov. 6, 2020

Related U.S. Application Data
(60) Provisional application No. 62/931,492, filed on Nov. 6, 2019.

(51) Int. Cl. A61B 90/00; G06T 11/00; A61B 8/00; A61B 5/00; A61B 8/08

(52) U.S. Cl. CPC A61B 90/36; G06T 11/00; A61B 8/4416; A61B 5/0077; A61B 8/463; A61B 8/5261; A61B 2090/502; A61B 8/565; A61B 8/486; A61B 8/488; A61B 8/4263; G06T 2210/41; A61B 2090/365; A61B 8/4472

(57) ABSTRACT

An example system includes an ultrasound probe device configured to provide a real-time ultrasound image and having a marker for visualization; an augmented reality (AR) device having a camera configured to provide a camera video output signal and a display configured to render an AR image from an AR video input signal; and a processor configured to: receive the camera video output signal and to extract localization information from the camera video output signal corresponding to the marker; receive the real-time ultrasound image; and combine the camera video output signal and the real-time ultrasound image to provide the AR video input signal.

### RELATED APPLICATION(S)

This application claims the benefit of U.S. Provisional Application No. 62/931,492, filed on Nov. 6, 2019. The entire teachings of the above application are incorporated herein by reference.

### BACKGROUND

Bedside, or point of care, ultrasounds are used by healthcare practitioners in a variety of settings and medical practice environments. However, even “portable” ultrasounds in most medical practices consist of a moveable cart the size of a shopping cart with a laptop, ultrasound probes and display screen attached. Each of these individual components are connected by a tangle of wires. The healthcare practitioner using a point of care ultrasound typically needs to stand on either side of a supine patient. Such setups are inefficient for the practitioner and the patient.

In addition, the practitioner is forced to divide their attention between the ultrasound probe on the patient and the laptop monitor displaying the medically meaningful ultrasound visualization, while also finding time to provide eye contact with the patient. This forced fragmentation of the practitioner's attention creates a suboptimal relationship with the patient, in addition to reducing the potential maximum efficiency with obtaining diagnostic ultrasound data or completing ultrasound guided procedures.

### SUMMARY

The present disclosure relates to a system that includes a pair of glasses with augmented reality functionality, a smartphone with augmented reality functionality (e.g., Apple iPhone with iOS 7.1+ or Google Android with 4.1+), and a wireless ultrasound transducer. System software enables compatibility and functionality for the wireless ultrasound transducer to transmit real-time ultrasound images to the smartphone and provide the option to overlay the images at a 1:1 scale onto a patient's body. This real-time visualization is displayed through the augmented reality lenses, while the smartphone can display a not-to-scale traditional ultrasound visualization.

In one embodiment, a system includes an ultrasound probe device configured to provide a real-time ultrasound image and having a marker for visualization. The system also includes an augmented reality (AR) device having a display and a camera configured to provide a camera video input signal. The system further includes a processor and a non-transitory memory device having processor instructions stored thereon, the instructions, when loaded, configuring the processor to receive the camera video input signal and to extract localization information from the camera video input signal corresponding to the marker, receive the real-time ultrasound image, and combine the camera video input signal and the real-time ultrasound image to provide an output video stream.

The output video stream may be an AR video output signal comprising the camera video input signal with the real-time ultrasound image overlaid thereon based on the extracted localization information, and the display may be a display screen configured to render an AR image from the AR video output signal. Alternatively, or in addition, the output video stream may be an AR video output signal comprising the camera video input signal with the real-time ultrasound image overlaid thereon based on the extracted localization information, and the display may be a display screen configured to render an AR image from the AR video output signal.

The rendered AR image may be positioned and aligned over an anatomically matching area of a subject based on the extracted localization information. Alternatively, or in addition, the rendered AR image may be positioned over a fixed portion of the display.

The ultrasound probe device may be configured to communicate with the processor over a wireless ultrasound application programming interface. The AR device may be configured to communicate with the processor over a wireless AR lens application programming interface.

The processor may be further configured to provide a sharable stream including the real-time ultrasound image to an Internet application or service. The sharable stream may include the camera video input signal. The Internet application or service may include capability for cloud storage, cloud processing, or live streaming. The sharable stream may be viewable by a receiving entity connected to the Internet application or service.

The processor may be further configured to issue commands to the ultrasound probe device, the commands including selections of M, B, and Doppler modes, and capture of still ultrasound images to be stored in the non-transitory memory device.

In another embodiment, a computer-implemented method for providing a combined video output signal includes providing a real-time ultrasound image via an ultrasound probe device having a marker for visualization. The method also includes providing a camera video input signal via an AR device having a display. The method further includes receiving, at a processor, the camera input video signal and extracting localization information from the camera video input signal corresponding to the marker. The method further includes receiving, at the processor, the real-time ultrasound image. The method further includes combining the camera video input signal and the real-time ultrasound image to provide an output video stream.

There are several advantages offered by embodiments in accordance with the present disclosure. First, interpretation of medical ultrasounds is difficult, except for the most anatomically adept physicians. Physicians undergo training to understand anatomy and can render three-dimensional models of human anatomy in their head, deconstructing and reconstructing these models into various two-dimensional representations. This visuospatial abstraction skill is acquired over a long course and can be curtailed by simply displaying the two-dimensional representations that are commonly seen in all forms of medical imaging and overlaying it with known three-dimensional structures. The system of the present disclosure does this and can expedite the time to proficiency in anatomical learning for healthcare practitioners. Second, the system of the present disclosure places the display directly in the field of view for the healthcare practitioner. A common problem that practitioners do not even know they have is that portable ultrasound displays currently have suboptimal viewing angles. The display is usually found on a mobile cart's laptop screen, either placed away from or behind the healthcare practitioner. Since portable ultrasounds necessitate the active and real-time use of an ultrasound probe to reveal a patient's anatomy, the focus of the healthcare practitioner is split between the ultrasound display and the ultrasound probe. This fragmentation of attention leads to poor eye contact with patients, time wasted in a patient-practitioner encounter and a higher barrier to skillful use. Third, the system of the present disclosure aims to utilize complete wireless functionalities. As ultrasound probes are commonly used in procedures that require maintaining a sterile field, having no wires attached to the probe allows for much easier, quicker and economical means of sanitizing the probe.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a rendering of a functional view through augmented reality lenses.

FIG. 1B is another rendering of a functional view through augmented reality lenses.

FIG. 2A illustrates elements of an example AR system.

FIG. 2B is a block diagram of an example AR system.

FIG. 3 illustrates a live view through augmented reality lenses of an ultrasound image overlaid onto a human subject.

FIG. 4 illustrates a smartphone with a user interface displaying the ultrasound image that is overlaid in FIG. 3.

FIG. 5A illustrates a smartphone displaying overlaid ultrasound and video images.

FIG. 5B illustrates a smartphone displaying simultaneous overlaid and individual ultrasound and video images.

FIG. 6 illustrates an example computer network, over which, embodiments of the claimed systems and methods may operate.

FIG. 7 is a system block diagram illustrating an example computer network, over which, embodiments of the claimed systems and methods may operate.

### DETAILED DESCRIPTION

A description of example embodiments follows.

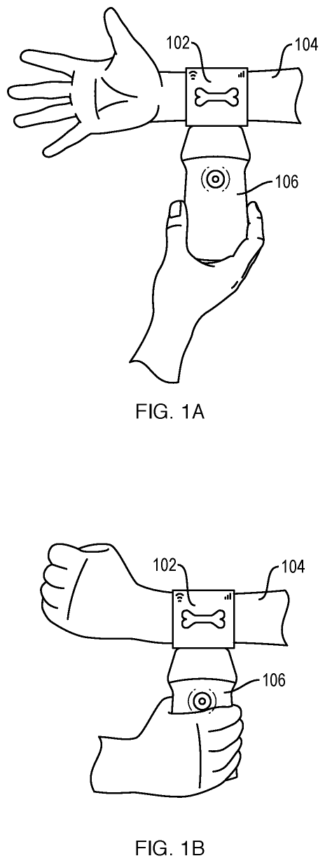

FIG. 1A illustrates the concept of the present disclosure, showing an augmented reality display 102 through lenses visualizing ultrasound images of the anterior side of a human patient's 104 forearm, taken with an ultrasound probe 106.

FIG. 1B illustrates the concept of the present disclosure, showing the same components of FIG. 1A visualizing ultrasound images of the posterior side of a human patient's 104 forearm.

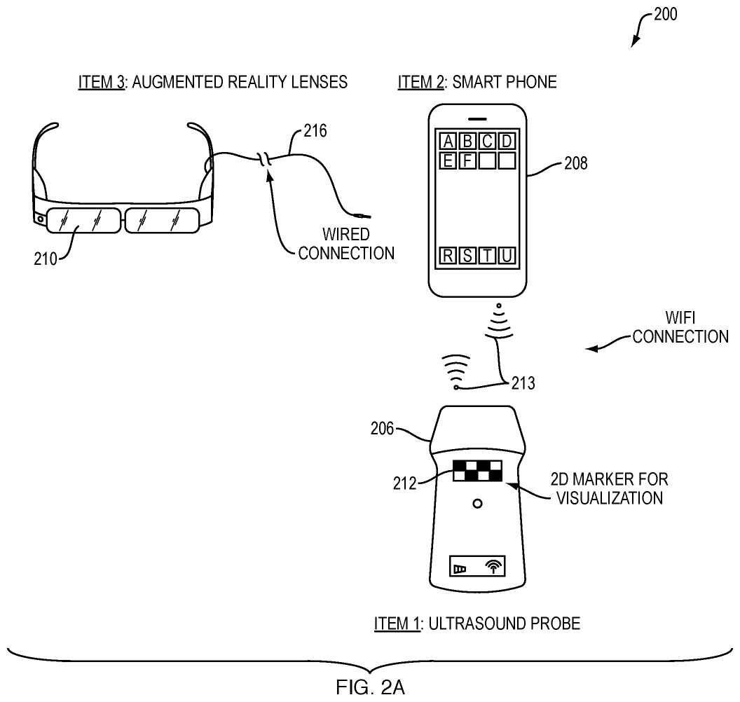

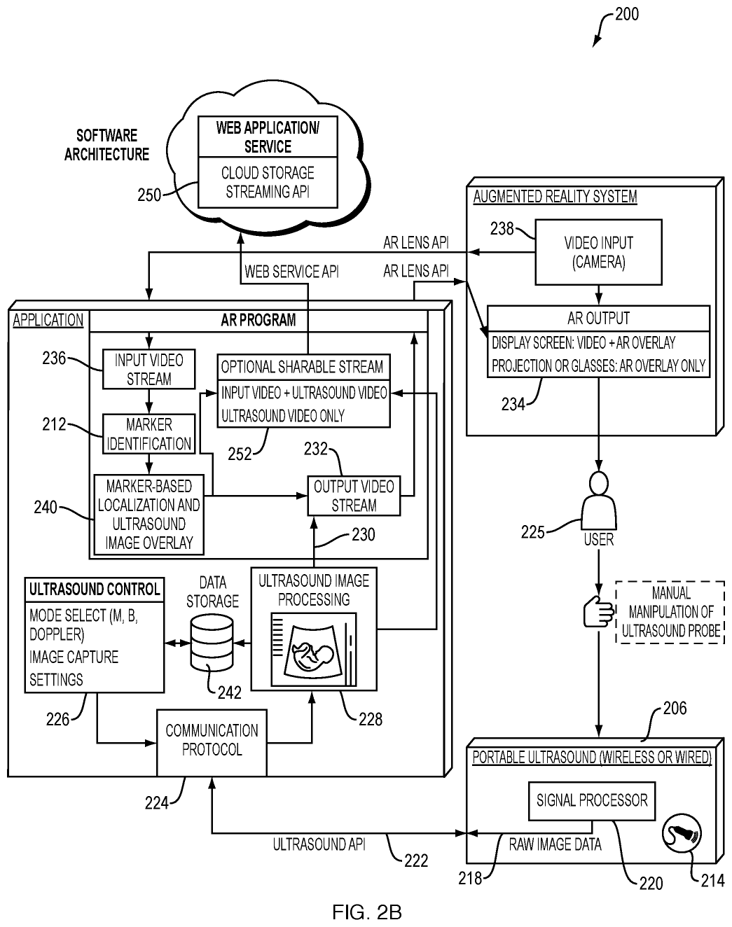

FIG. 2A illustrates an example AR system 200 that includes:

Item 1: Wireless ultrasound probe 206

Item 2: Smartphone 208

Item 3: Augmented reality lenses 210

Item 1 is a wireless ultrasound probe 206 that can house internal and external components. Internally, there can be a piezoelectric ultrasound transducer 212, analog to digital signal converter, wireless transmitter 224 (using WPA 2.4 GHz and 5 GHz channel transmission) and battery. There can be a single, centrally located button on the exterior of the ultrasound encasement that allows for functional interaction with the software and device operability.

Externally, there can be a unique identifying marker 214, a USB type-B female port for charging, and an LED screen that displays Wi-Fi connectivity and battery level.

Item 2 is a smartphone 208 with augmented reality capabilities (e.g., Android 4.1 or higher, iOS 9.1 or higher).

Item 3 is a pair of augmented reality lenses 210.

Item 1 may communicate with Item 2 via a wireless connection 213 including wireless 2.4 GHz or 5 GHz transmission protocols 224.

Item 2 may communicate with Item 3 via wired connection 216.

Item 3 may communicate with Item 2 by direct, augmented reality computer visualization of the unique two-dimensional identifying marker 212.

Reference is now made to FIG. 2B, which illustrates a block diagram of the AR system 200.

The piezoelectric ultrasound transducer 214 may emit and receive ultrasound pulses via a multiplex channel system. The raw, analog data may be converted to a digital signal comprising raw image data 218 via the internal signal processor 220 comprising an analog-to-digital converter. This digital signal 218 may then be sent across an ultrasound application programming interface (API) 222 through wireless transmission communication protocols 224 from Item 1 to Item 2. However, the wireless communication may be a 2-way channel and Item 2 can also send ultrasound control commands 226 to the internal components of Item 1, allowing for variation in pulse frequency and amplitude.

Item 2 may leverage a smartphone's 208 central processing unit 228 to process the digitally converted signal into medically relevant, two-dimensional ultrasound images 230. An output video stream 232 comprises these ultrasound images 230, culminating in an AR output 234. In some embodiments, Item 2 can display the ultrasound images 230 with a graphical user interface, allowing it to switch between two display modes on Item 3. In an embodiment, the output video stream 232 includes the ultrasound images 230 overlaid upon an input video stream 236. The input video stream 236 may be generated as a camera video input signal 238 trained upon the ultrasound probe 220 and the area of the patient 104 being imaged by the ultrasound probe 220.

Item 3 may detect and spatially localize 240 Item 1's orientation via the unique identifying marker 212. In addition, the device's software may be configured to place the AR output 234 comprising the ultrasound images 230 in an anatomically relevant position and overlay the images 230 on the patient, which can be visualized through Item 3. There can be two display modes on Item 3, one where the ultrasound images 230 match the patient's 104 anatomy to scale (mode 1), and one where the images 230 do not match to scale (mode 2).

Mode 1 (deformable anatomical registration with image superimposition) has been previously mentioned above but will be elaborated further. Using deformable registration, the ultrasound images 230 may be placed and oriented anatomically over a patient 104 using computer vision three-dimensional modeling of human anatomy. Mode 1 allows for visualization of ultrasound images 230 with respect to a patient's 104 anatomy.

Mode 2 displays the ultrasound images 230 locked on Item 3's screen, irrespective of Item 1's position and orientation or the patient's 104 position and orientation. Mode 2 allows for visualization and manipulation of size and positioning of the display irrespective of a patient's 104 anatomy.

The software components enable a variety of functions for point of care ultrasound use. Through a graphical user interface on Item 2, the user 225 can issue ultrasound control commands 226 to enable and switch between B mode, M mode, phased array, and color doppler. These variations in ultrasound functionality are produced by the software telling the hardware in Item 1 to vary the intensity (or amplitude) or frequency of pulse emission and detection. Color doppler can be detected solely through utilization of doppler shifts or detection of velocity changes in relation to time. Distances and on-screen measurements can be made using an algorithm that determines two-dimensional measurements, based on the determined pulse frequency and amplitude. Data can be stored on the user's 225 smartphone 208 (item 2)'s internal hard drive 242, e.g., as .jpg for images and .mkv files for video. In some embodiments, the patient 104 and the user 225 may be the same person, and in some embodiments, the patient 104 and the user 225 may be different persons, such as when the user 225 is a doctor, a technician, or other healthcare practitioner.

In an example operation of the system 200, Item 1 is turned on using the central button with a short-press (e.g., <3 seconds). Next, the Wi-Fi network or wireless connection 213 on Item 1 is now discoverable by smartphones 208. Using Item 2's built-in Wi-Fi connectivity, the user 225 finds and connects to Item 1's Wi-Fi network 213 using a unique identifier code and password. Next, Item 3 is plugged into Item 2 via USB Type-C connectivity. Then, the smartphone app is opened in Item 2 to display an interface that visualizes the ultrasound images 232. The system software automatically enables connectivity with Item 3 and visualizes the ultrasound images 232 in an augmented reality (AR) output 234.

To enable the piezoelectric transducer 214 and visualize real-time ultrasound scanning, the central button may be short pressed (<3 seconds). Finally, a long press of Item 1's button may turn the device off, shut off the Wi-Fi network 213, and disable any active connections between Item 2 and Item 3.

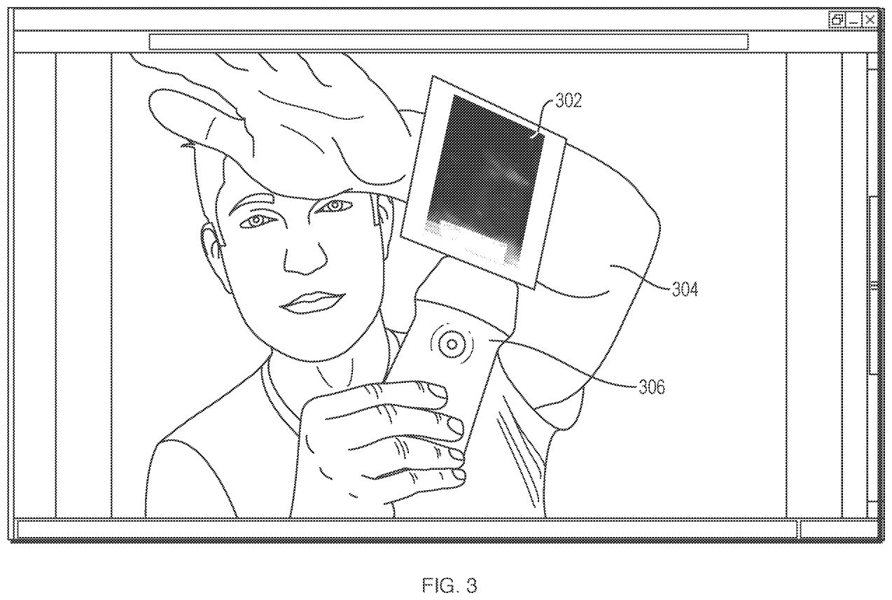

FIG. 3 depicts an embodiment of the present disclosure, showing an augmented reality display 302 through lenses visualizing ultrasound images of a human patient's 304 arm, taken with an ultrasound probe 306.

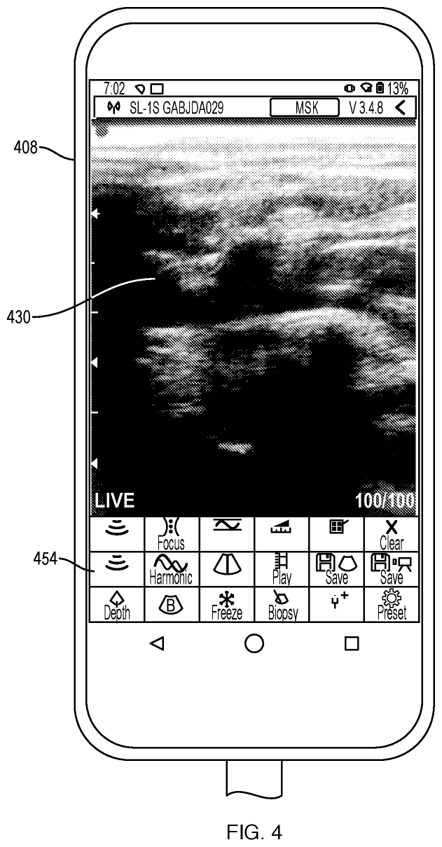

In an example embodiment, the system software was developed in Unity version 2019.4 with a Vuforia version 9 computer vision API. An example Item 1 is an OEM wireless linear 7.5 MHz ultrasound probe as shown in FIG. 3, an example Item 2 is a Google Pixel 1 smartphone as shown in FIG. 4, and an example Item 3 is a pair of Epson Moverio BT-3000 augmented reality glasses.

The marker based augmented reality identification piggybacks through Vuforia's 2D image based marker recognition software.

FIG. 4 depicts an embodiment showing a smartphone 408 with ultrasound images 430 displayed within a graphical user interface 454.

By eliminating the need for continuously searching for an optimal viewing angle of the portable ultrasound display, the user 225 can utilize both hands for manual manipulation and procedural interventions. Additionally, by having the ultrasound display correlate with the probe's 206 orientation, the time to proficiency can be expedited by eliminating the need for highly proficient visuospatial re-orientation. Less skilled users like technicians can provide the same value procedure to patients without sparing safety. Also, by eliminating the need to deconstruct and reconstruct a two-dimensional slice of a patient's 104 anatomy, a healthcare practitioner can use ultrasounds as a meaningful way to engage their patients. With a direct overlay of their ultrasound image onto their own body, patients can see where the ultrasound images are being taken.

With the device being wireless, sterile fields can easily be maintained by encasing the entire ultrasound probe 206 in a plastic cover-slip.

With the device utilizing an individual's smartphone 208 as the central signal processing unit 228, hardware and software components can be upgraded individually throughout the lifetime of the device. This aims at reducing overall healthcare spending while also allowing for incremental improvements in technology to find its way to patients and provide increased value at the same incremental steps.

Optional elements include a wireless component that connects Item 2 to Item 3 to improve the portability of the system overall. Another optional element is the ability to utilize 5G cellular connectivity, to increase the bandwidth of transmittable data and speed of transmission. Further optional enhancements include databasing and image indexing.

In other embodiments, the display may be maintained without a visualized marker 212, either through multiple accelerometer registration, or computer-aided vision for object recognition. This functions by taking the individual frames captured in a video for pixelated analysis. For example, individual pixel analysis of RGB values can generate data for each pixel in a given frame. This RGB data can then be converted to grayscale, and first order, second order, and third order radiomic data can be analyzed to give intrinsic properties to identified objects. By identifying the ultrasound transducer probe 206 as an object, it will have uniquely registered radiomic data for a combination of its RGB pixel information, as well as skewness, kurtosis, range and positioning of grayscale images with respect to known shading algorithms.

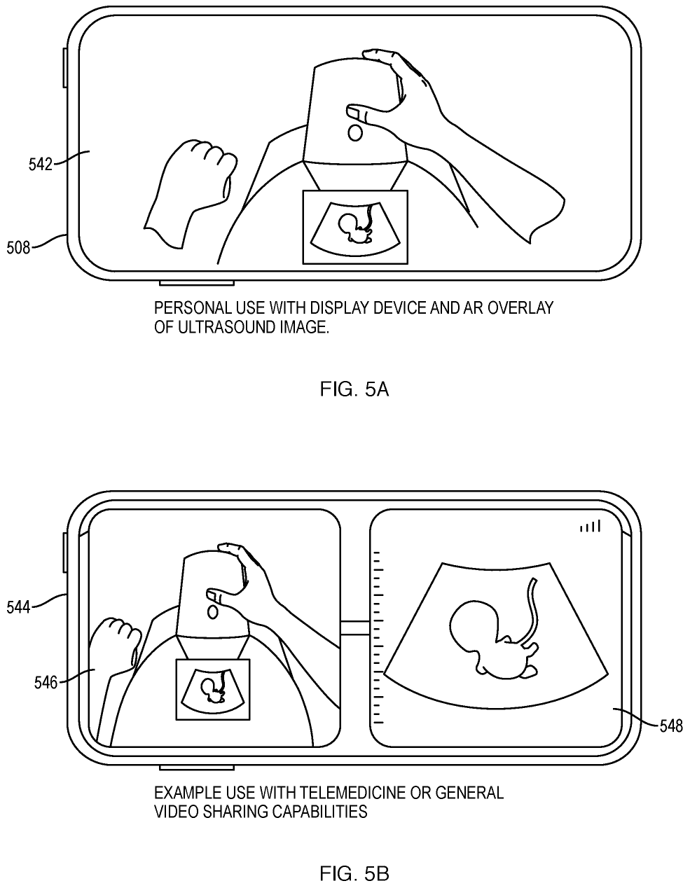

Additionally, the augmented reality lenses 210 may be replaced by any combination of a video input device, video output device and central processing unit. Instead of lenses or glasses, this may be a mirror with the video output as a self-reflective and/or two-way mirror with the input as a camera placed somewhere on the mirror. Or, this could be extrapolated to a laptop or smartphone 508 with the video input as its onboard camera and the output as the display screen 542, as shown in FIG. 5A.

FIG. 5B depicts an embodiment that uses a separate video monitor 544 to simultaneously display two versions of the output video stream 232. A first output video stream version 546 may comprise the ultrasound images 230 overlaid upon the input video stream 236, while a second output video stream version 548 may include the ultrasound images 230 alone. Alternatively, either the first 546 or second 548 output video stream versions may include the input video stream 236 alone. Arrangements such as these offer the advantage of a simultaneous patient view and a practitioner view as the image still floats in place to help the patient position the device to display what the practitioner wishes to show the patient. This can be particularly useful in telemedicine situations. In addition, the display may be recorded which can also allow for building in alerts for trouble spots.

Furthermore, the individual components of and relating to the input video stream 236, the central processing unit 228, the output video stream 232, and ultrasound image input data 218 can be compacted into myriad combinations. For example, the augmented reality lens 210 may harbor the central processing unit 228 and eliminate the smartphone 208 entirely; this would leave the system to two separate hardware components, the ultrasound transducer 206 and the augmented reality lens 210 with the central processing unit 228. Alternatively, the central processing unit 228 can be harbored in the ultrasound transducer 206, eliminating the smartphone 228 entirely. It should be noted that a wired connection from the ultrasound probe to the central processing unit is an expected variant.

Returning now to FIG. 2B, utilizing a web application or web service 250 could enable cloud storage for data such as image data. Furthermore, utilizing a web application or web service 250 could enable cloud computing to circumvent the need for local image processing to allow for complete computer-aided vision. Therefore, this would eliminate the need to have a smartphone 208 to act as the central processing unit 228 and allow for much higher rates of real-time data analysis. This could prove fruitful in radiographically identifying clinically meaningful structures, such as pathology for diagnostic purposes, or normal anatomy for procedural guidance. The web application or web service 250 could accept an optional sharable stream 252 from the central processing unit 228. The optional sharable stream 252 could include ultrasound images 230. The optional sharable stream could further include the input video stream 236.

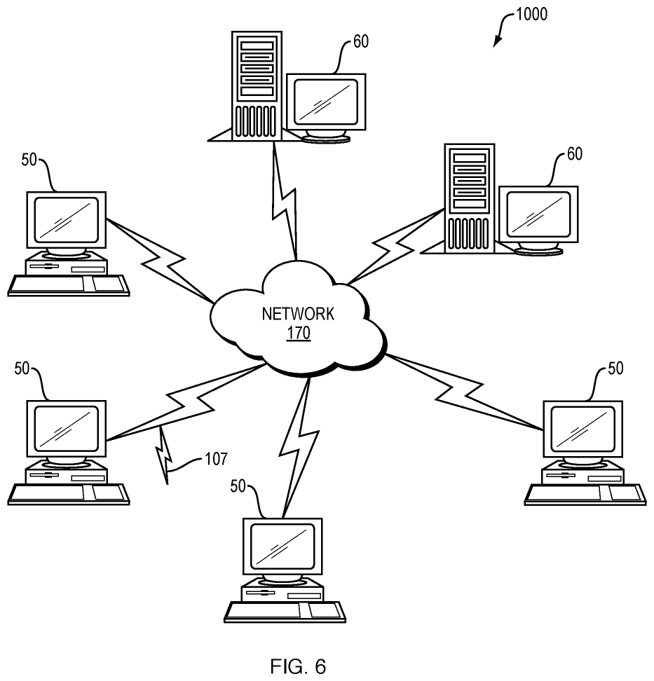

FIG. 6 illustrates a computer network (or system) 1000 or similar digital processing environment, according to some embodiments of the present disclosure. Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

Client computers/devices 50 may be configured with a computing module (located at one or more of elements 50, 60, and/or 70). In some embodiments, a user may access the computing module executing on the server computers 60 from a user device, such a mobile device, a personal computer, or any computing device known to one skilled in the art without limitation. According to some embodiments, the client devices 50 and server computers 60 may be distributed across a computing module.

Server computers 60 may be configured as the computing modules which communicate with client devices 50 for providing access to (and/or accessing) databases that include data associated with target objects and/or reference objects. The server computers 60 may not be separate server computers but part of cloud network 70. In some embodiments, the server computer (e.g., computing module) may enable users to determine location, size, or number of physical objects (including but not limited to target objects and/or reference objects) by allowing access to data located on the client 50, server 60, or network 70 (e.g., global computer network). The client (configuration module) 50 may communicate data representing the physical objects back to and/or from the server (computing module) 60. In some embodiments, the client 50 may include client applications or components executing on the client 50 for determining location, size, or number of physical objects, and the client 50 may communicate corresponding data to the server (e.g., computing module) 60.

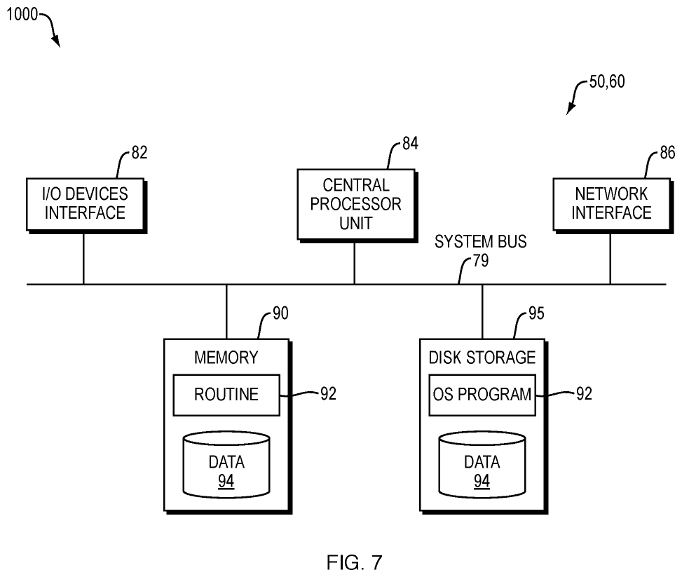

Some embodiments of the system 1000 may include a computer system for determining location, size, or number of physical objects. The system 1000 may include a plurality of processors 84. The system 1000 may also include a memory 90. The memory 90 may include: (i) computer code instructions stored thereon; and/or (ii) data representing ultrasound images or input video data. The data may include segments including portions of the ultrasound images or input video data. The memory 90 may be operatively coupled to the plurality of processors 84 such that, when executed by the plurality of processors 84, the computer code instructions may cause the computer system 1000 to implement a computing module (the computing module being located on, in, or implemented by any of elements 50, 60, 70 of FIG. 6 or elements 82, 84, 86, 90, 92, 94, 95 of FIG. 7) configured to perform one or more functions.

According to some embodiments, FIG. 7 is a diagram of an example internal structure of a computer (e.g., client processor/device 50 or server computers 60) in the computer system 1000 of FIG. 7. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 6). Memory 90 provides volatile storage for computer software instructions 92 and data 94 used to implement some embodiments (e.g., input and output video streams described herein). Disk storage 95 provides non-volatile storage for computer software instructions 92 and data 94 used to implement an embodiment of the present disclosure. A central processor unit 84 is also attached to the system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the present disclosure. The computer program product 92 can be installed by any suitable software installation procedure, as is well known in the art. In another embodiment, at least a portion of the software instructions may also be downloaded over a cable, communication and/or wireless connection. Other embodiments may include a computer program propagated signal product 107 (of FIG. 6) embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the routines/program 92 of the present disclosure.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

Embodiments or aspects thereof may be implemented in the form of hardware (including but not limited to hardware circuitry), firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

While example embodiments have been particularly shown and described, it will be understood by those skilled in the art that various changes in form and details may be made therein without departing from the scope of the embodiments encompassed by the appended claims.

What is claimed is:

1. A system comprising:
an ultrasound probe device configured to provide a real-time ultrasound image and having a marker for visualization;
an augmented reality (AR) device having a display and a camera configured to provide a camera video input signal;
a processor and a non-transitory memory device having processor instructions stored thereon, the instructions, when loaded, configuring the processor to:
receive the camera video input signal and to extract localization information from the camera video input signal corresponding to the marker;
receive the real-time ultrasound image; and
combine the camera video input signal and the real-time ultrasound image to provide an output video stream.

2. The system of claim 1 wherein:
the output video stream is an AR video output signal comprising the camera video input signal with the real-time ultrasound image overlaid thereon based on the extracted localization information; and
the display is a display screen configured to render an AR image from the AR video output signal.

3. The system of claim 1 wherein:
the output video stream is an AR video output signal comprising the real-time ultrasound image; and
the display is a projection or AR glasses configured to render an AR image from the AR video output signal.

4. The system of claim 3 wherein the rendered AR image is positioned and aligned over an anatomically matching area of a subject based on the extracted localization information.

5. The system of claim 3 wherein the rendered AR image is positioned over a fixed portion of the display.

6. The system of claim 1 wherein the ultrasound probe device is configured to communicate with the processor over a wireless ultrasound application programming interface.

7. The system of claim 1 wherein the AR device is configured to communicate with the processor over a wireless AR lens application programming interface.

8. The system of claim 1 wherein the processor is further configured to provide a sharable stream including the real-time ultrasound image to an Internet application or service.

9. The system of claim 8 wherein the sharable stream further includes the camera video input signal.

10. The system of claim 8 wherein the Internet application or service includes capability for cloud storage, cloud processing, or live streaming.

11. The system of claim 8 wherein the sharable stream is viewable by a receiving entity connected to the Internet application or service.

12. The system of claim 1 wherein the processor is further configured to issue commands to the ultrasound probe device, the commands including selections of M, B, and Doppler modes, and capture of still ultrasound images to be stored in the non-transitory memory device.

13. A computer-implemented method for providing a combined video output signal, the method comprising:
providing a real-time ultrasound image via an ultrasound probe device having a marker for visualization;
providing a camera video input signal via an AR device having a display;
receiving, at a processor, the camera input video signal and extracting localization information from the camera video input signal corresponding to the marker;
receiving, at the processor, the real-time ultrasound image; and
combining the camera video input signal and the real-time ultrasound image to provide an output video stream.

14. The method of claim 13 further comprising rendering an AR image from the combined video output signal on the display, wherein:
the output video stream is an AR video output signal comprising the camera video input signal with the real-time ultrasound image overlaid thereon based on the extracted localization information; and
the display is a display screen.

15. The method of claim 13 further comprising rendering an AR image from the combined video output signal on the display, wherein:
the output video stream is an AR video output signal comprising the real-time ultrasound image; and
the display is a projection or AR glasses.

16. The method of claim 15 further comprising positioning and aligning the rendered AR image over an anatomically matching area of a subject based on the extracted localization information.

17. The method of claim 15 further comprising positioning the rendered AR image over a fixed portion of the display.

18. The method of claim 13 further comprising configuring the ultrasound probe device to communicate with the processor over a wireless ultrasound application programming interface.

19. The method of claim 13 further comprising configuring the AR device to communicate with the processor over a wireless AR lens application programming interface.

20. The method of claim 13 further comprising configuring the processor to provide a sharable stream including the real-time ultrasound image to an Internet application or service.

21. The method of claim 20 wherein the sharable stream further includes the camera video input signal.

22. The method of claim 20 wherein the Internet application or service includes capability for cloud storage, cloud processing, or live streaming.

23. The method of claim 20 wherein the sharable stream is viewable by a receiving entity connected to the Internet application or service.

24. The method of claim 13 further comprising configuring the processor to issue commands to the ultrasound probe device, the commands including selections of M, B, and Doppler modes, and capture of still ultrasound images to be stored in the non-transitory memory device.
